(19) United States
(12) Patent Application Publication
(10) Pub. No.: US 2007/0009468 A1
(43) Pub. Date: Jan. 11, 2007

(54) Photodynamic method of inducing the growth of hair

(76) Inventor: Eric F. Bernstein, Gladwyne, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/431,531

(22) Filed: May 11, 2006

Related U.S. Application Data
(60) Provisional application No. 60/680,034, filed on May 12, 2005.

(51) Int. Cl. A61K 48/00; A61K 8/73; A61K 8/64

(52) U.S. Cl. 424/70.13; 514/44; 424/70.14

(57) ABSTRACT

Disclosed herein is a method of inducing the growth of hair on the skin of a human, such as the scalp, the method comprising systemically or topically altering the hormonal status of the skin, such as with androgen blockers or estrogen-like hormones, and exposing the skin to at least one light source prior to or simultaneous with the altering.

This application claims the benefit of domestic priority to U.S. Provisional Patent Application Ser. No. 60/680,034, filed May 12, 2006, which is herein incorporated by reference in its entirety.

The present disclosure is related to a method of growing hair on the skin of a human by exposing the skin to at least one electromagnetic source, such as a light source, subsequent to or simultaneous with a process for altering the hormonal status of the skin.

Focused light energy, such as laser energy, has become increasingly popular to remove unwanted hair from the body. For example, laser hair reduction utilizing pulsed red, or infrared laser radiation is currently used to target melanin pigment in unwanted hair, stimulating its removal. Most hair removal lasers and light sources further utilize a cooling device to extract heat from the epidermis, providing relative protection to melanin-containing surface epidermis, increasing the relative amount of heat delivered to the hair follicle versus the epidermis.

Although the initial strategy for laser hair removal focused on destroying hair by targeting the melanin pigment contained within the hair shaft and in the surrounding keratinocytes thereby producing enough heat to destroy the cells of the hair follicle, the actual mechanism behind laser hair removal is much more complex. Rather than being a simple physics problem of targeting pigmented structures and heating them until they or adjacent structures are destroyed, laser hair removal relies on a complex set of biological events following laser treatment, including a localized inflammatory response around the hair shaft. For these reasons, photonic energy, such as laser treatment, has not typically been associated with inducing hair growth.

Contrary to conventional thinking, the present disclosure is related to a method of inducing the growth of hair on human skin by exposing the skin to at least electromagnetic source, such as a light source, subsequent to or simultaneous with a process for altering the hormonal status of a human.

It is understood that the process for altering the hormonal status of the skin may be performed by administering a composition to the human systemically, topically, or a combination of both. In one embodiment, the composition may comprise at least one hormone, receptor blocker, receptor agonist, or RNA derivative.

Systemically altering the hormonal status of the skin may comprise the intravenous, oral, intramuscular, or subcutaneous administration of at least composition in an amount sufficient to change the hormonal status of the skin.

In various embodiments, the at least one composition can be in the form of a lotion, gel, or spray. The at least one composition may also be found in or on a transdermal patch. In this embodiment, the transdermal patch may be transparent to certain wavelengths of light, which allows for a process that simultaneously alters the hormonal status of the skin while exposing the skin to at least one light source.

Another embodiment is directed to a method of inducing the growth of hair on a human head, which comprises applying to the human head at least one androgen blocker or estrogen-like hormone to form a treated area, and subsequently exposing the treated area to electromagnetic radiation, such as light.

The following definitions are used herein:

RNA—Ribonucleic acid. A long chain polymer of genetic material similar to DNA except that it is usually found in a single-stranded form rather than as a double helix, it is made up of the ribo nucleotides A, G, C, and U (instead of T), it is somewhat less resistant to cellular degradation enzymes than DNA, and it functions most often as a transient messenger of genetic information rather than as a permanent store-house as is the case for DNA.

RNAi—RNA interference. A natural cellular mechanism by which RNA is recognized as “foreign” due to its existence in a double-stranded form. This results in the degradation of the double-stranded RNA, along with single-stranded RNA having the same sequence.

As used herein “RNA derivative” is intended to encompass the following:

dsRNA—Double-stranded RNA. RNA with two strands instead of the typical one.

siRNA—short interfering RNA. An intermediate in the RNAi process in which the long double-stranded RNA has been cut up into short (˜21 nucleotides) double-stranded RNA. The siRNA stimulates the cellular machinery to cut up other single-stranded RNA having the same sequence as the siRNA.

siNA—short interfering nucleic acid. The same thing as siRNA, except that it may or may not contain ribonucleic acid (RNA) residues.

In one embodiment, the present disclosure is related to a method of inducing the growth of hair comprising applying a topical medication that can affect the hormone status of skin or produce a hormone-like effect, such as testosterone or estrogen to the area where hair growth is desired. Non-limiting examples of such topical medications include creams, solutions or gels comprising minoxidil (sold under the tradename Rogaine®), DHEAS, or DHEA cream.

In this embodiment, a pre-treatment period ranging, for example, from a few days to a few weeks may used to build up the topical hormone in the deeper layers of the skin. Then an electromagnetic source such as an intense pulsed light, laser, radiofrequency source, a high or low intensity light-emitting diode, or other electromagnetic radiation source is used to treat the area where the hormone cream has been applied. Repeated treatments may be necessary to induce noticeable hair growth. Maintenance treatments may also be necessary to maintain hair growth.

In another embodiment, the topical composition may comprise a component that decreases the effect of estrogen-like hormones in the skin, such as a cream, ointment solution or gel containing an estrogen-receptor blocker followed by an electromagnetic source such as an intense pulsed light, laser, radiofrequency source, a high or low intensity light-emitting diode, or other electromagnetic radiation source is used to treat the area where the estrogen-blocking cream has been applied. Repeated treatments may be necessary to induce noticeable hair growth. Maintenance treatments may also be necessary to maintain hair growth.

In one embodiment, the method can be used to induce hair growth in areas that typically lose hair in response to androgenic hormones, such as the scalp. This embodiment comprises applying androgen blockers or estrogen-like hormones to the skin, which is subsequently treated with electromagnetic radiation. For example, testosterone, DHEA, DHEAS, estrogen blockers or combinations thereof are applied to the scalp, which is subsequently treated with a source of electromagnetic radiation, such as visible light or infrared radiation or radio frequency (RF). Other sources of light that may be used include any known pulsed laser commonly used to remove hair. Light can also be applied using intense pulsed light, photodynamic therapy using photosensitizers, such as 5-ALA plus light, intense pulsed light plus radio frequency, or using radio frequency alone.

In yet another embodiment, the present disclosure is related to a method of inducing the growth of hair comprising the systemic, e.g., oral ingestion, of medications or supplements that alter hormone status. Non-limiting examples of such medication include those that limit or reduce the level of testosterone or estrogen in the area to be treated. For example, medicines that reduce and/or prevent at least one enzyme, such as the 5-alpha reductase enzyme, from being converted into dihydrotestosterone (DHT), the main hormone linked to hair loss, that can be taken orally include the family of proscars, such as those sold under the tradename Propecia® and Avodart®. Other examples of oral medications that can have a similar affect are those that decrease the estrogen levels of the area of skin to be treated. After taking the described oral medication, the desired area of skin is exposed to the previously described electromagnetic radiation, which includes an intense pulsed light, a laser, a radio frequency source, a high or low intensity light-emitting diode, or other electromagnetic radiation source. In another embodiment, the patient may be treated with topical and/or oral agents that block affect the effective hormone responsiveness or status of the skin such as siRNAs to block estrogen receptors, gene therapy to alter the hormone status or hormone responsiveness of the skin such as genes to induce or suppress dihydroxytestosterone levels in the skin. Following such treatment, the skin is exposed the previously described electromagnetic radiation.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention.

Other embodiments of the invention will be apparent to those skilled in the art from consideration of the specification. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of inducing the growth of hair on the skin of a human, said method comprising altering the hormonal status of the skin and exposing the skin to at least one light source prior to or simultaneous with said altering.

2. The method of claim 1, wherein said altering the hormonal status of the skin is performed by administering a composition to the human systemically, topically, or a combination of both.

3. The method of claim 2, wherein said composition comprises at least one hormone, receptor blocker, receptor agonist, or RNA derivative.

4. The method of claim 2, wherein systemically altering the hormonal status of the skin comprises the intravenous, oral, intramuscular, or subcutaneous administration of at least composition in an amount sufficient to change the hormonal status of the skin.

5. The method of claim 2, wherein topically altering the hormonal status of the skin comprises applying to the skin at least one composition in an amount sufficient to change the hormonal status of the skin.

6. The method of claim 5, wherein the at least one composition is in the form of a lotion, gel, or spray.

7. The method of claim 5, wherein the at least one composition is in or on a transdermal patch.

8. The method of claim 7, wherein said transdermal patch is transparent to certain wavelengths of light, and said process comprises simultaneously altering the hormonal status of the skin and exposing the skin to at least one light source.

9. The method of claim 1, wherein the at least one light source is chosen from laser light, intense pulsed light, diode light, fluorescent light, chemi-luminescent light, and incandescent light, light-emitting diodes.

10. The method of claim 9, wherein the laser light has a wavelength ranging from 600 to 1200 nm.

11. A method of inducing the growth of hair on the human head, said method comprising applying to the human head at least one composition that comprises androgen blockers or estrogen-like hormones to form a treated area, and subsequently exposing the treated area with electromagnetic radiation.
